(19) United States
(12) Patent Application Publication
(10) Pub. No.: US 2009/0136599 A1
(43) Pub. Date: May 28, 2009

(54) HOMEOPATHIC COMPOSITION AND KIT THEREFORE

(76) Inventor: Miria De Amorim, Rio De Janeiro (BR)

Correspondence Address: DUANE MORRIS LLP - Philadelphia;IP DEPARTMENT, 30 SOUTH 17TH STREET, PHILADELPHIA, PA, 19103-4196, US

(21) Appl. No.: 12/296,970

(22) PCT Filed: Apr. 13, 2007
(86) PCT No.: PCT/BR2007/000097

§ 371 Date: Oct. 13, 2008

(30) Foreign Application Priority Data
Apr. 13, 2006 (BR) PI0601496-8

(51) Int. Cl. A61K 33/04

(52) U.S. Cl. 424/705

(57) ABSTRACT

The present invention refers to homeopathic medicine compositions, developed from seven chemical elements which are submitted to successive homeopathic dilutions by means of a specific process in order to provide precise sequence for administration to the patient within given time intervals. The present invention also refers to medicinal kits containing medicinal compositions and to a method for the administration of the medicinal compositions in accordance with the present invention.

### FIELD OF THE INVENTION

The invention refers to homeopathic medicine compositions, developed from seven chemical elements which, when submitted to successive homeopathic dilutions, provide precise sequence for the administration to the patient within given time intervals.

The invention also refers to a method for administration the medicinal compositions for the treatment of any clinical profile within all specialties of medical clinics, under the same curing action also in veterinary clinics.

Furthermore, the invention refers to kits to make said medicinal compositions available.

### BACKGROUND OF THE INVENTION

Homeopathics may be regarded as a complex medical system, including doctrine, semiology, diagnosis and therapeutics, being available as specific medical rationality, although sharing the anatomy and physiology of modern medicine.

There are three basic principles or doctrines distinguishing homeopathics from other systems or therapies. These principles are:

(i) individualization, including drug selection by using the principle of the law of similars;
(ii) promotion of ego-cure by using the minimal dosage of a drug; and
(iii) use of all symptoms to evaluate curing standards.

This model of medical knowledge was created by Chistian Friedriech Samuel Hahnemann (1755-1843), a progressive phylosopher who, after stopping to exercise medicine for being unhappy with alopathic therapeutic behavior used at that time, opted to translate medical books. While translating medical articles written by Cullen, he disagreed with the understanding supplied by the Scottish doctor on the medicinal action of quina, then used to treat intermittent fevers. According to the author, its action would be related to its bitter and aromatic properties.

Hahnemann then tested the drug in himself and, from then on, he collected for years extense notes on the effects caused by different elements of nature (mineral, vegetal and animal) to himself and to other healthy men. He concluded that said toxic effects, as in the case of quina, would exactly correspond to the set of symptoms that drug would be able to cure. Therefore, the therapeutic ideal of homeopathics settled on the search and finding of simillimum, the medicine which, having produced an artificial disease in a healthy man, would be the medicine to be prescribed when the symptoms of a natural disease are exactly the same as that artificial disease.

This concept would form the first basic class of the “Homeopathic Doctrine”, based on the curing principle by similars Similia Similibus Curantur, a principle which was initially disclosed by Hypocrates in the 5^{th} Century (460-350 b. C.), brought again by Paracelsus in the 16^{th} Century and consolidated by Hahnemann when he created homeopathics in 1796.

The model of homeopathic knowledge then started to be created by Hahnemann, initially from references of experiments in healthy men. Such references have then been progressively coded, classified and organized according to idiopathic symptoms, originating medicinal pathogenesis, which set is found in books of homeopathic medical matter. By organizing data, he created a method which, instead of being deductive and logical, aims to be predominantly experimental and, in therapeutical intervention, besides being empirical and arbitrary, dares to enter into questions related to the primary causes of diseases.

In his classic book, “Organon of Medicine: The Rational Art of Healing”, initially published in 1810, Hahnemann discloses the basic principles of said medical system indicating as an object of therapeutics the sick individual and not the disease, now seeing man as an undissociable integrity. Causes of diseases would then be in the inside of human beings, which would need to be qualified to make the morbid process understandable.

Healing power, according to Hahnemann, manifests with the lowest possible dosage of the dynamized medicine, produced by means of the homeopathic dynamization process, consisting in releasing unknown physical properties from the matter in dilution from a certain kind of shaking called succussion. It has currently been underlined that the basic effect of high dynamization consists of energy delocation by means of resonance interaction between transmitter (organism) and receiver (dilution).

Hahnemann remarked that, under higher doses, clear symptoms were significantly changed and the return to good heatlh was much slower.

He then started to dilute medicines, worried about toxicity and also to avoid the appearance of very unpleasant symptoms. However, Hahnemann had already concluded that diseases are a change in vital energy and, upon dilution, he was lowering the concentration of active principles. He concluded that he needed a way to compensate, then giving the medicine the dynamization process, which are succussions, then allowing to release the medicinal power of each substance.

As the time passed, he noticed that the more the initial substance was diluted, the better results were obtained, thus minimizing the appearance of unpleasant symptoms, more quickly reverting to equilibrium, to a healthy state.

Therefore, the principles governing homeopathic therapeutics are not based on the chemical properties of the substances, but rather in physical properties of high dilutions, now proved by huge scientific research.

Said energy released by the substance is resonant with the vital field of individual, thus promoting vital equilibrium and providing a cure.

Homeopathics also reflects the importance of experimentation in healthy men, since it avoids the interference of foreign symptoms disturbing the evaluation of true pathogenetic symptoms.

Principles and laws of homeopathics have been conceived in a vitalist vision, understanding that life is the result of an action over matter from something foreign to it, of non material and non measurable nature, but which we notice exists in living organisms and does not exist in non-living substances. According to vitalism, said harmony as observed in living bodies, the condition of life itself, is due to vital energy or vital principle (force). It is present throughout the body, distributed among feelings and functions.

According to Hahnemann: “The totality of symptoms in this profile of disease essence reflected outwards, i.e. the affection of the vital force should be the main and only means by which the disease informs the required medicine—the only means to determine the choice of the most appropriate medicine—in summary, the totality of symptoms.”

A disease would be nothing more than a disorder of the vital force. If a man gets sick by means of dynamic influence, he should also be healed by means of a similar dynamic process. “Therefore, the dynamic action of morbific influences in a healthy man, as well as the dynamic force of medicines in the vital principle to restore health is nothing else than an infection, which is not material by any means and not mechanical or aestethical by any means.”

All the basic assumptions of homeopathics as mentioned above have been experimentally proven within biomedical investigation criteriae from cell and molecular biology, concluding that cell self-recovery is stimulated when a substance is given in low quantity, based on the law of similars.

Homeopathic medicines are derived from the vegetal kingdom, the animal kingdom, imponderable, synthetic products, dynamized alopathic medicines, homeopathic preparations themselves, biotherapeuticals—which are medicines produced from secretions, excretions, tissues, organs of healthy or pathological animals or vegetables, or even from organisms holding disease principle, microorganisms, nosodiums—which are medicines prepared from pathological secretion or excretion products from animals or vegetables, sarcodiums—prepared from physiological secretions from animals or vegetables, self-nosodiums—prepared from secretions, pathological products or parts of organs or tissues which must containg the pathological principle responsible for the sickness state of a living body, to be used in its own healing, and organotherapeuticals—prepared from organs.

The reality of homeopathical handling is based on the analysis and definition of Centesimal, Decimal and Fifty Millesimal scales, as well as the description and step by step detailing of preparation methods for derived pharmaceutical forms: Hahnemannian Method (soluble and insoluble drugs), Korsakovian Method and Continued Flow Method.

Homeopathics select medicines by comparing detailed drug profiles for medicines and the full profile of the symptoms of a sick person. This selection process is called the principle of simillimum, sometimes written in Latin as Simila similibus curentur, meaning “concept of healing law”.

Generally speaking, from its origins, homeopathics were based on three basic principles: treatment by the principle of similars, experiments in healthy men and use of dynamized medicines (infinitesimal dosage). Hahnemann stated that each individual, upon becoming sick from any morbity, becomes sick according to his/her biological, psychic, familiar, sexual, behavior, characteristic etc. history. To become sick is at first a biological, existential and social question of vulnerability. Symbolic and psychological aspects of the subject cannot be ignored.

Homeopathics, now recognized as a medical specialty in 14 countries, was recognized in Brazil in 1980. On that decade, the Federal Government recognized pharmacopoeia, offering to physicians and patients products with relatively low cost and low toxicity. As opposed to drugs as used by alopathic medicine, acting directly over the physiopathological processes as related to the symptoms of the disease, homeopathic medicines promote the improvement of the general state of health of an individual, by stimulating his/her immunological system upon bringing up appropriate responses for each situation. Therefore, homeopathic treatment allows the individual to re-establish health and prevent diseases, not however producing side effects as experienced by many conventional treatments.

Homeopathic medicines have been more and more used under various situations, such as for the treatment of stomatitis after bone marrow transplant, to accelerate cicatrization, in changes of the threshold of pain, in the reduction, incidence and progression of cancer in animals.

However, if the homeopathic fact is a concrete reality, it becomes necessary to prove it from research as made by the so-called official science, under defined criteriae and clearly outlined protocols for testing. Homeopathics may then be able to build a new theory which, by disclosing scientific character, may create basis for its practice.

The homeopathic therapeutical model is frequently criticized for being based on different assumptions from the classical scientific knowledge, with repeated references to the non-existence of reliable scientific evidence to prove the efficacy of homeopathic treatment of diseases, or also that homeopathic assumptions are pseudoscientific arguments, and an evidence-based medicine is required within the context of scientific rationality. Said requirement has generated, over the state of the art of homeopathics, attention directed to basic research, since scientific features of a research are related to the use of methods and techniques allowing to reproduce experiments and their analysis, i.e. they should be able to generate general and faithful formulations on the states of the world or, states of perception of the real world.

Upon reflection on the theme, the question of the importance of a therapeutical study directly turned to the totality of symptoms of a disease is raised, not ignoring causes and adjacent circumstances, but bearing in mind, after summing all these symptoms, what should really be treated within the patient, covering mental, emotional and physical aspects.

From the homeopathic point of view, even if the clinical profile includes symptoms from various ethyologies, the method as used allows to specifically order various groups of specific symptoms for later application of compatible medicines to the totality of symptoms.

Homeopathic medicine has been notably recognized within the last few decades for the search of new therapeutical solutions for medical practice. With this new opening, medical knowledge has been enlarged and enriched with new concepts and criteriae, offering the impulse given by research in the biological field to find ways to represent important forms of hope and relief for human beings.

Homeopathic theory is based on phenomena and on a qualitative model which has always received severe criticism in its history for not opening space to suffer empirical contrast. This has been due to the ad hoc supposition of individual sensitivity making theory practically unaccessible to refutation, always alleging that related individuals are not sensible to substances.

To solve this question of individual sensitivity, the Applicant has searched, by means of clinical experience for more than twenty years, to develop research on a homeopathic therapeutical model which, according to the law of similars, promoted regulation of the biological field. Said assumption was based on the possibility of primary and direct actuation over said biological matrix, presented as a forming field from which different disorder models classified as diseases emerge. This conception differs from classical homeopathic studies which, by the law of similars, act secondarily, requiring for that absolute specificity among homeopathic medicines as used and each one of these uncountable emerging morbid standards composing different pathologies.

Said biological matrix or field, known by physicists as the “fifth field” and by biologists as the “biofield”, represents, according to B. Goodwin, in Development and Evolution, Journal of Theoretical Biology, 97-1982, interaction between biological fields acting over existing organic units and integrating the basic unit of the form and organization of the living systems. Physicists disclose such fifth field as synergic and having an organizing effect; as a field fulfilling all the space, penetrating and permeating everything and presenting the property to reconnect objects just like they were connected in the past. In Biology, A. Gurwitsch in The Metting of Science and Spirit, in White, J. Paragon House, New York, 1990, searching for data as observed in embriogenesis, presented said matrix as a morphogenetic field (form generator), establishing itself as a non material force field determining at last the role of individual cells, their properties and their relations with neighboring cells.

The biofield is therefore configured as a standard, i.e. one quantic dimension depending on the order, rhythm, frequency, flow, resonance and synchronism. In the emerging theory of living systems, life process is disclosed as the continued incorporation of autopoietic organization standards in a physical dissipative structure. This process, according to H. Matutana and F. Varela in Autopoiesis and Cognition, D. Reidel, Dordrecht, The Netherlands, 1980, was identified as a cognitive process, since it synthesizes all the organizing activity of living bodies in all levels of life, as a mental process.

From this standard, different configurations incurred from all kinds of information received by the individual along his/her life are made. This information directly interacts with such standard composing the biofield and may be of chemical nature as in the case of intoxications; biological nature as in case of infectocontagious diseases; physical nature by exposition to different radiations; or even psychic information such as trauma or any class of stress. Depending on the morbid potential of said information as received, the individual may suffer such deviation in his biofield, losing biological memory regarding universal standards compatible with health. From this point, he needs for his recovery new coherent information, so to re-program said matrix for self-organization. The big characteristic of chronic diseases is exactly the loss of biological memory over said health-compatible standards. Individuals, even before a simple picture, frequently have their defense system blocked and their whole biological system allows the disease to progress.

In this context, a new homeopathic model has been developed by the Applicant from mapping certain substances from nature, which properties allow to establish coherent resonance regarding this biofield. Said elements have been compiled throughout the years, composing a mapping study concerning the employed order, flows and specific frequencies, so that the synergic dynamics between these elements may exert the principle of similars concerning different morbid standards which may eventually emerge from that biological terrain.

Homeopathic medicines send, in fact, biophysical information to the biofield. In this context, the effectiveness of that information does not depend on the presence of molecules in the employed solution. We should highlight that the higher the morbid potential of information received in the biofield, the higher should be the infinitesimal dilution of homeopathic medicines as employed. This happens because, in the biophysical conception, inversely to what occurs with biochemical conception, the higher the homeopathic dilution, the higher the dynamic potential of said substances.

To develop said new “epistemologic profile”, the proposal to equation another point of critical support concerning homeopathics was studied which, according to P. M. C. Lourenco in Homeopatia, Ciência ou Ficgao? Meta Análise da Teoria Homeopática, a dissertation presented to obtain a Master Degree in Public Health in the Area of Epidemiologics Concentration, FIOCRUZ, 1989, had not as yet solved the main problem stimulating its construction: the ellaboration of a principle unifying therapeutics. From this assumption, research was made towards a model which is able to possibly regulate said biological terrain which, according to H. Labout in L'action biologique comportamentale et physiopathologique, Ed. Paris, Ed. Masson, 1986, would cover its genetic command—the notion of neuro-endocryne, metabolic history of the individual and the reply to every aggressive agent, even in acute pathology, showing the existence of biological memories and the importance of interactive systems in the functioning of the human body.

Chronic affections represent the reply to information which is sent to the biofield which, mentioning as an example chronic intoxications, represent chemical information which, at the level of said biological matrix, erases the biological memory concerning coherent health standards in the individual. This is the cause of irreversibility of chronic symptoms of individuals suffering said intoxications.

This condition requires therapeutics operating by means of biophysical information at the level of said biological terrain, so to allow the system to receive new information which is coherent with health standards. This allows reintegration and later reorganization of said biological system towards looking for its internal homeostasis. Homeopathic therapeutics are included in this context, establishing possible prevention and control not only of different classes of dysfunctions and disorders, but also showing the intrinsic property of homeopathic medicines, so to enable the reorganization of said biological matrix, so to reguide said biofield towards its internal homeostasis. This consequently shows its action over patients and not specifically the disease, which ends up to explain the wide range of actuation of said homeopathic medicinal preparation for different pathologies.

A possibility of a new glance to the official medicine concerning the question of information at biological field level is therefore evidenced, thus making therapeutic actuation over the dimension of individual susceptibility become possible. Due to the complex environmental factors progressively causing impact to the health of individuals, thus promoting even more complex systemic disorders, a discussion is raised concerning the need of therapy acting directly over said biological matrix by means of biophysical information, so to re-guide this self-organization standard towards health-compatible standards.

Therefore, a series of studies conducted in the field of basic research, so to show, by means of experiments as presented in said works, that the homeopathic medicinal composition object of the invention is able to offer coherent and positive replies, in the relation between employed dosages and observed symptoms, so to open up space for its use as alternative therapeutics in acute and chronic affections, as well as all kinds of intoxications. This possibility is proved by means of the results as disclosed in basic research inserting repetitive condition into the experiments, thus showing high degree of empirical confirmation, i.e. deductive support, a required virtue for the scientific hypothesis to be accepted.

The discussion on the perspective of new paradigms for homeopathics and the replies obtained from the disclosed experiments for basic research confirm the theory on the regulation of the biological field over clinical evaluations of holistic medicines which should be based on a global health coverage and comprised as a dynamic equilibrium method.

Within this context, the purpose of the homeopathic treatment is not to specifically remove or supress symptoms, as conventional therapeutical methods do, but rather to restore the equilibrium of the organism as a whole. Homeopathics is therefore a methodological specialty in the field of medical therapeutics, aiming to treat patients with their diseases, not only prioritizing the disease, as emphasized by current medical practices.

Homeopathics is therefore a treatment system using especially prepared and highly diluted substances to widely put into action the healing devices of the body itself. Due to high dilution, it does not present toxic effects, producing low or no side effects.

As disclosed by the patent application BR 9502977-0 of the same Applicant, homeopathic medicinal compositions as developed from chemical elements are already known, which are submitted to successive homeopathic dilutions, providing, by means of precise sequence for administration to the patient within given time intervals, significant improvement in patient's disease over alopathics.

The present invention improves homeopathic medicinal compositions as presented in the state of the art by using new dynamization ranges and precise sequences for administration of the composition in given time intervals, so to reach better and faster results for patients, besides providing the same healing action in veterinary clinics.

### SUMMARY OF THE INVENTION

It is therefore the object of the present invention to provide homeopathic medicine compositions, developed from chemical elements which, when submitted to successive homeopathic dilutions, provides precise sequence to cure various diseases.

Another object of the present invention is the use and a method for the administration of homeopathic medicinal compositions in accordance with specific characteristics in the sequence of the administration of components, precise intervals between each component and which may be given to patients once or twice until the end of the treatment.

It is also an object of the present invention to provide kits comprising said composition with presentation structure, posology and application for different pathologies.

### BRIEF DESCRIPTION OF THE DRAWINGS

These and other objects, enhancements and effects of the present invention will be evident to the experts in the art from the attached schematic figures, in which:

FIG. 1—illustration of a first embodiment of the closed medicinal kit (1) of the present invention.

FIG. 2—illustration of the medicinal kit as shown by FIG. 1, in an open position, showing the perspective view of the support (2) as folded.

FIG. 3—enlarged perspective view of the support as shown by FIG. 2 as folded.

FIG. 4—perspective view of the support as shown by FIGS. 2 and 3 as open, showing the location of the 25 hermetically closed recipients (3).

FIG. 5—front view of the support as shown by FIGS. 2 to 4, showing the location of the 25 hermetically closed storage elements in their sequence of administration (4) and the time interval for administration between each medicine (5).

FIG. 6—illustration of a second embodiment of the closed medicinal kit (10) of the present invention.

FIG. 7—illustration of the medicinal kit as shown by FIG. 6, in an open position, showing the way of attachment of storage elements (13) of each one of the 25 medicines.

FIG. 8—front view of the kit as shown by FIGS. 6 and 7 as open, showing the location of the 25 storage elements (13), their sequence of administration (14) and the time interval for administration between each medicine (15).

### DEFINITIONS

Some definitions are still required to better understand the present invention:

MEDICINE: all the substances with therapeutical properties to which living organisms are sensible in some way and degree. A substance which may combat a disease, curing or alleviating such a disease. For the patient, medicine is only the substance which ended up by curing him after administration. Medicines are active substances, compositions and other manifestations able to heal someone, even those which are non material or unexplainable, as a surprise, cosmic or climatic alterations.

HOMEOPATHIC MEDICINE: substance presumed or applied as a medicine, which had been tested in healthy men, therefore having no pathogenesis. Not all homeopathic medicines, however, were tested in healthy men. Some of them are empirical, with ethiological similarity, such as nosodiums.

DYNAMIZATION: process to release latent dynamic medicinal power from active substances to men.

SUCCUSSION: shaking a medicine after each dilution. It is done in pure Hahnemannian homeopathic techniques, by shaking a ⅔ full flask vertically and beating it against a semi-resilient hard shield, approximately 30 cm high.

POWER: final result of each step of the dynamization and dilution process.

CENTESIMAL SCALE (CH): dilution prepared under 1:100 (active ingredient: inert ingredient) proportion.

DECIMAL SCALE (DH): dilution prepared under 1:10 (active ingredient:inert ingredient) proportion. This scale was introduced into Homeopathics by Hering.

FIFTY MILESIMAL SCALE (LM): the derived form will follow the 1:50,000 proportion.

Hahnemannian Method:

Centesimal and Decimal Scale—Soluble Drugs: basic pharmaceutical form (TM or previous dynamization), inert ingredient ethanol under different gradients, manual or mechanical succussion and dilution process; Insoluble Drugs: insoluble drugs, when their solubility is lower than 10% in the liquid inert ingredient, and any drug for the preparation of LM, lactose for the solid phase and ethanol under different gradients for the liquid phase, grounding process for the solid phase, manual or mechanical dilution and succussion for the liquid phase.

Fifty Milesimal Scale—mineral or biological, vegetal or animal, drug, fresh whenever possible, inert ingredient lactose for the solid phase and ethanol under different gradients for the liquid phase, grounding process for the solid phase, manual or mechanical dilution and succussion for the liquid phase.

Korsakovian Method:

30 CH medicine in 70% ethanol, inert ingredient 70% ethanol for intermediaries and 30% for dispensing, manual or mechanical dilution and succussion process.

Continuous Flow Method:

30 CH medicine in 70% ethanol, inert ingredient water (obtained by distillation, bidistillation, deionization-sterilizing filtering), dilution process and mechanical vortexing.

### DETAILED DESCRIPTION OF THE INVENTION

Based on research and all concepts as shown by the background of the present invention, new homeopathic medicinal compositions select only seven elements of nature among 190 elements used in homeopathics and which, when applied in specific homeopathic dilution and sequence and given within precise time intervals, provide quick and effective healing, acting both for chronic and acute clinic states and with application in all specialties of medical clinics, with the same curing action in veterinary clinics.

The seven essential elements as used in the composition of the present invention may be designated by their universal Latin nomenclature:
Antimonium crudun: homeopathic medicine prepared from antimonium sulphide (SB₂S₃);
Kali carbonicum: homeopathic medicine prepared from potassium carbonate K₂CO₃);
Mercurius solubilis: homeopathic medicine prepared from a preparation of mercury nitrate (Hg(NO₃)₂);
Sulphur: homeopathic medicine prepared from sulfur (S);
Natrum muriaticum: homeopathic medicine prepared from sodium chloride (NaCl);
Aurum metallicum: homeopathic medicine prepared from gold (Au);
Ammonium muriaticum: homeopathic medicine prepared from ammonia chloride (NH₄Cl).

The present invention establishes epistemiologic studies in the domains indicated by Quantic Physics concerning the self-organizing standards with the main glands of the endocrine system, now related to different consensual conscience centers:
Antimonium crudun: predominant action over hypophysis or pituitary gland;
Kali carbonicum: predominant action over the pineal gland;
Mercurius solubilis: predominant action over the thyroid gland;
Sulphur: predominant action over suprarenal glands;
Natrium muriaticum: predominant action over thymus gland;
Aurum metallicum: predominant action over gonads;
Ammoniuim muriaticum: predominant action over coccygeal gland.

Homeopathic medicinal compositions of the present invention start from the isolated dynamization of each substance composing them, under any homeopathic power as used by the present invention, according to the skills as established by the Brazilian Homeopathic Pharmacopoeia, obtaining dilution of the prepared substance under hydroalcohol concentration which may vary between 1% and 99%, with 30% hydroalcohol solution preparation being preferably chosen, for having ideal alcohol content to preserve homeopathic medicine, being therefore used to conventionally soak globules and tablets.

Dilutions as used by the present invention are preferably obtained from the following proportions of homeopathic elements (self-organizing factors):

a) Antimonium crudun under 30% hydroalcohol solution to obtain 100% diluted solution; Kali carbonicum under 30% hydroalcohol solution to obtain 100% diluted solution; Mercurius solubilis under 30% hydroalcohol solution to obtain 100% diluted solution; Sulphur under 30% hydroalcohol solution to obtain 100% diluted solution; Natrum muriaticum under 30% hydroalcohol solution to obtain 100% diluted solution; Aurum metallicum under 30% hydroalcohol solution to obtain 100% diluted solution; Ammonium muriaticum under 30% hydroalcohol solution to obtain 100% diluted solution.
b) 0.2 ml parcel is taken from each solution of step a) and transferred to the recipients to be hermetically closed.

The present invention refers to medicinal compositions containing seven specific homeopathic elements, i.e. Antimonium crudun, Kali carbonicum, Mercurius solubilis, Sulphur, Natrum muriaticum, Aurum metallicum and Ammonium muriaticum, under dynamization ranges between 6 D and 50 D, 6 CH and 999 CH, 6 LM and 200 LM or 1 MFC and 60 MMFC (continued flow) under 30% hydroalcohol solution to obtain 100% diluted solution.

Preferably, homeopathic elements may be repeated under medicinal compositions of the present invention.

More specifically, the present invention refers to medicinal compositions containing seven specific homeopathic elements which are repeated in a given order, for a total of 25 components under the following concentrations:
1) 0.20 ml of the substance Antimonium crudun in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
2) 0.20 ml of the substance Kali Carbonicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
3) 0.20 ml of the substance Mercurius solubilis in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
4) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
5) 0.20 ml of the substance Natrum muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
6) 0.20 ml of the substance Aurum metallicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
7) 0.20 ml of the substance Ammonium muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
8) 0.20 ml of the substance Mercurius solubilis in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
9) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
10) 0.20 ml of the substance Natrum muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
11) 0.20 ml of the substance Antimonium crudun in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
12) 0.20 ml of the substance Kali Carbonicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
13) 0.20 ml of the substance Mercurius solubilis in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
14) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
15) 0.20 ml of the substance Natrum muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
16) 0.20 ml of the substance Aurum metallicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
17) 0.20 ml of the substance Ammonium muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
18) 0.20 ml of the substance Mercurius solubilis in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
19) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
20) 0.20 ml of the substance Natrum muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
21) 0.20 ml of the substance Antimonium crudun in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
22) 0.20 ml of the substance Kali Carbonicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
23) 0.20 ml of the substance Mercurius solubilis in dynamizations within the following ranges: from 6 D to 50 D, from 6 CH to 999 CH, from 6 LM to 200 LM or from 1 MFC to 60 mMFC (continued flow) under 30% hydroalcohol solution to obtain 100% diluted solution.
24) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
25) 0.20 ml of the substance Natrum muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.

According to the interaction flow between endocrine centers, we may divide the composition into groups as follows:

Group 1:

Antimonium crudum and Kali carbonicum

Group 2:

Mercurius solibilis, Sulphur and Natrum muriaticum

Group 3:

Aurum metallicum and Ammonium muriaticum.

From the above groups, we may define that the compositions of the present invention comprise the following groups, ordered under the following sequence:

group 1, group 2, group 3, group 2, group 1, group 2, group 3, group 2, group 1, group 2, successively, summing up 25 components.

The compositions of the present invention promote the balance of the biological field, by fixing healing standards with individual susceptibility, so to quickly and mildly develop internal homeostasis by using Hahnemannian dilutions, i.e. Decimal, Centesimal, Fifty Milesimal and Continued Flow, under the powers from 6 D to 50 D, from 6 CH to 999 CH, from 6 LM to 200 LM or from 1 MFC to 60 MMFC (continued flow).

Preferably, the compositions of the present invention have powers from 6 D to 50 D or from 6 CH to 60 MMFC.

All components of the present invention are presented under two different medicinal powers for each used kit, and dimerization evalution may be effected according to each specific clinical case.

Medicinal compositions of the present invention may be produced in one or more forms such as globules (sucrose granules), tablets (lactose), liquids (hydroalcohol or water solution) and also in powder (lactose packaged in small envelopes). Preferably, due to the problems existing with medicines given in liquid form, homeopathic globules, preferably microglobules or microspheres, have been chosen for technical preparation, with mass from 3 mg to 5 mg, made of saccharin and lactose with another adjuvant. Inert globules are soaked for thirty minutes with the homeopathic medicine already under the desired dilution are left to dry at a temperature of about 40° C.

The compositions in accordance with the present invention, under the powers from 6 D to 50 D, from 6 CH to 999 CH, from 6 LM to 200 LM or from 1 MFC to 60 MMFC (continued flow), given within a shorter time interval between the components of the same group and longer between one group and another, are used for the treatment of any clinical profile within all human and animal medical specialties.

Under the above cited conditions, a few examples of disorders and dysfunctions treated by the compositions in accordance with the present invention can be mentioned, but with no limitation. They are serious profiles such as Zoster herpes, hepatitis C with very high viral load, collagenosis, rheumatic profiles, Parkinson, hypothyroidism and hyperthyroidism, prostatic hyperplasia, ulcerative rectum colitis, tendinitis gravis, repeated effort injuries, migraines, neuropathies, encephalitis, fibromyalgias, atopic dermatitis, asthma, bronchitis, serious eczema, gastric ulcera, duodenitis, hepatitis B, hepatitis A, gastroesophagus reflow, strong painful profiles, genetic origin diseases such as Huntington's disease, kidney insufficiency, severe constipation profiles, acute and chronic diarrheas, urinary infections, rhinitis, sinusitis, bad digestion, non-specific virosis, bacterial infections, cardiovascular diseases, blood hypertension, tropical diseases, non-specific allergic profiles, amygdalitis, otitis, sinusitis, conjunctivitis, bronchial asthma, gynecological diseases, endocrine dysfunctions, circulatory dysfunctions, respiratory dysfunctions, emotional profiles with deep depression and psychiatric diseases, agrochemical intoxication profiles, reactive depression profiles, dysthymia, loss of libido, prostration, apathy, memory dysfunctions, anxiety, obsessive compulsive dysfunctions and phobia profiles such as panic syndrome.

The following pathologies and symptoms treated by the compositions of the present invention may also be mentioned as examples. They are tonsil abscess, vascular accident, acne, adenitis, amygdalitis, falciform anemia, anorexia nervosa, anxiety, arthritis, arthrosis, infant autism, histeric throat bolus, bronchospasm, bronchopneumonia, bronchiolitis, bronchitis, repetition chalazion, cystitis, pilonidal cyst, irritable colon, cattarhal child with repetition infections, acute cough crisis, allergic crisis, asthma crisis, intervertebral hernia crisis, depression, prostration and sleeplessness, diabetes, difficulties of speech and anti-social behavior, dysthymia, Charcot-Marie-Tooth's disease, cervical pain, sciatic pain, chronic pain, pain in the front region, joint pain, retina druses, scarlatina, calcaneus spur, strophulus, pharyngitis, fever, fibromyalgia, anal fissure, stammer, gastroenteritis, glaucoma, cold, hematometrium, genital herpes, hyperactivity, hypertention, relapsing HPV, repetition higher aerial route infection, aerial route infection, urinary infection, sleeplessness, labyrinthitis, laringitis, systemic erythematosis lupus, mastitis, blood dejection, mamary microcalcifications, mononucleosis, preeclampsia, prostatitis, psoriasis, idiopathic trombocytopenic purpura, laryngeal/pharyngeal reflow, retinoblastoma, rhinosinusitis, rotavirus, chronic hoarseness, cerebral vascular accident sequels, Bell's paralysis sequels, syphilis, panic syndrome, myasthenic syndrome, hip transitory synovitis, allergic cough, acute tracheitis, urticaria, virosis, HTLV1 virus and vomiting.

Preferably, serious profiles such as Zoster herpes, hepatitis C with very high viral load, collagenosis, rheumatic profiles, Parkinson, hypothyroidism and hyperthyroidism, prostatic hyperplasia, ulcerative rectum colitis, tendinitis gravis, repeated effort injuries, migraines, neuropathies, encephalitis, fibromyalgias, atopic dermatitis, asthma, bronchitis, serious eczema, gastric ulcera, duodenitis, hepatitis B, hepatitis A, gastroesophagus reflow, strong painful profiles, genetic origin diseases such as Huntington's disease and kidney insufficiency are treated by using medicinal powers from 6 D to 100 CH, even more preferably under 9 D power.

Preferably, specific cases of chronic and serious profiles such as collagenosis and degenerative diseases are treated by using compositions of the present invention under medicinal powers of 6 D or 10 CH.

Preferably, specific cases of migraines, cephalea and pain profiles in general are treated by using medicinal powers of 6 D, 9 D, 30 D and 10 CH.

Preferably, profiles of severe constipation, acute and chronic diarrheas, urinary infections, rhinitis, sinusitis, bad digestion, non specific virosis, bacterial infections, cardiovascular diseases, blood hypertension, tropical diseases, non specific allergic profiles, amygdalitis, otitis, sinusitis, conjunctivitis and bronchic asthma are treated by using medicinal powers from 9 D to 15 MFC, even more preferably by using power from 155 CH to 15 MFC, particularly 5 MFC.

Preferably, specific cases of acute and infectious profiles are treated by using medicinal powers of 6 D or 50 D, 5 MFC or 10 MFC; and 6 CH or 500 CH.

Preferably, specific cases of chronic and acute profiles such as bronchic asthma, rhinitis and sinusitis are treated by using medicinal powers from 6 D to 50 D, 6 CH or 500 CH and from 1 MFC to 15 MFC, particularly 12 D.

Preferably, emotional profiles with serious depression and psychiatric diseases are treated by using medicinal powers from 9 D to 15 MFC, even more preferably by using power from 9 D to 50 D, particularly 12 D.

Preferably, for agrochemical intoxication profiles, medicinal power from 9 D to 155 CH is used.

Preferably, for reactive depression profiles, dysthymia, loss of libido, prostraction, apathy, memory dysfunctions, anxiety, obsessive compulsive dysfunctions, phobia profiles such as panic syndrome are treated under medicinal powers from 9 D to 60 MMFC, preferably from 9 D to 500 CH, particularly at 12 D.

Preferably, specific cases of non-specific virotic profiles are treated by using medicinal powers from 9 D to 10M, preferably 5M.

Preferably, specific cases of gynecological diseases are treated by using medicinal powers from 9 D to 5 MFC.

Preferably, specific cases of blood hypertension are treated by using medicinal powers from 9 D to 200 CH.

Preferably, specific cases of endocryne dysfunctions are treated by using medicinal powers from 6 CH to 5M.

Preferably, specific cases of circulatory dysfunctions are treated by using medicinal powers from 6 D to 150 CH.

Preferably, specific cases of respiratory dysfunctions are treated by using medicinal powers from 9 D to 200 CH.

The present invention also refers to the use of homeopathic medicinal compositions for the production of a homeopathic medicine in which the medicinal power is defined in accordance with the specific clinic cases.

The present invention also has as another object a method for treatment against human and animal disorders and dysfunctions.

Furthermore, the present invention also has as its object at least two embodiments of kits (1, 10) containing the medicinal composition of the invention, comprising a total of 25 storage elements (3, 13).

Said storage elements (3, 13) are disposed in alternate groups with two and three elements each, representing the above defined groups as a function of the interaction flow between endocrine centers.

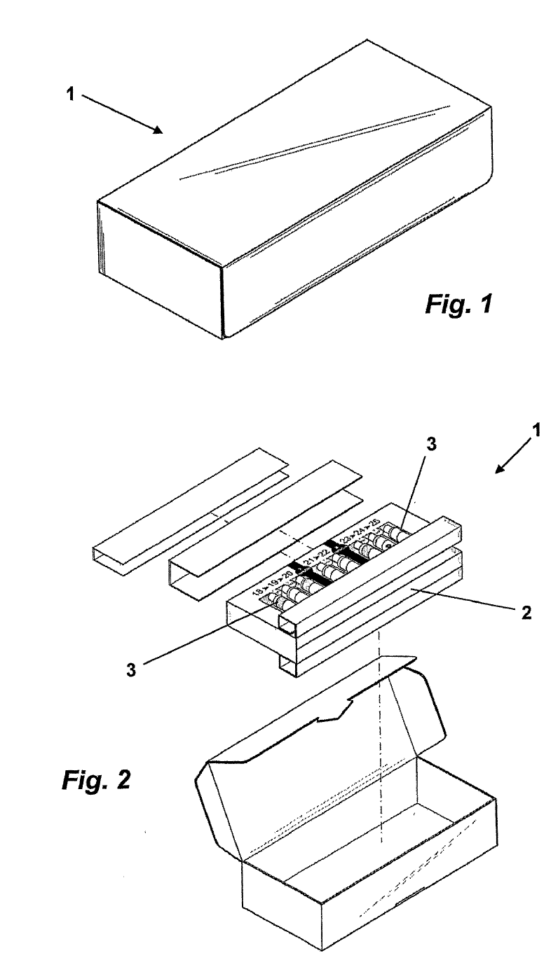

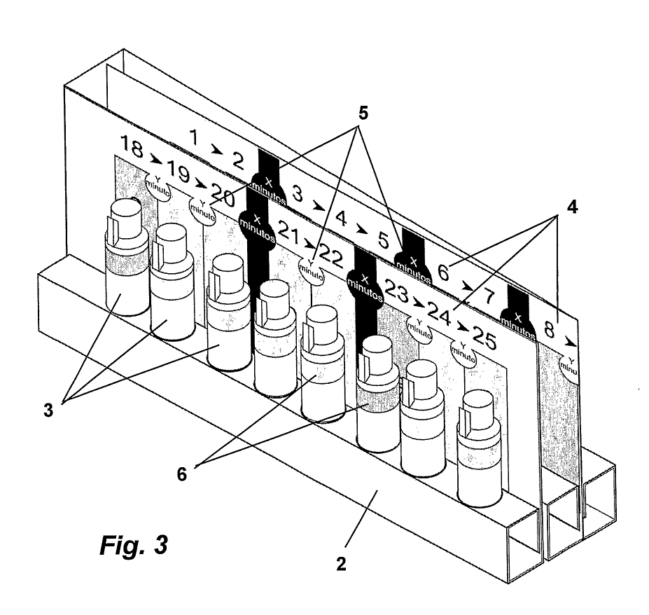

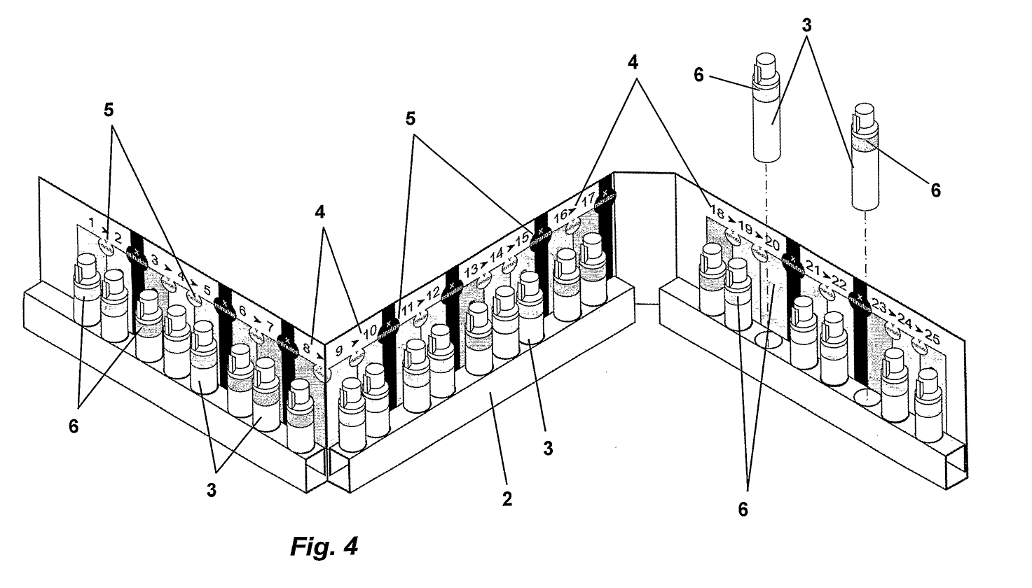

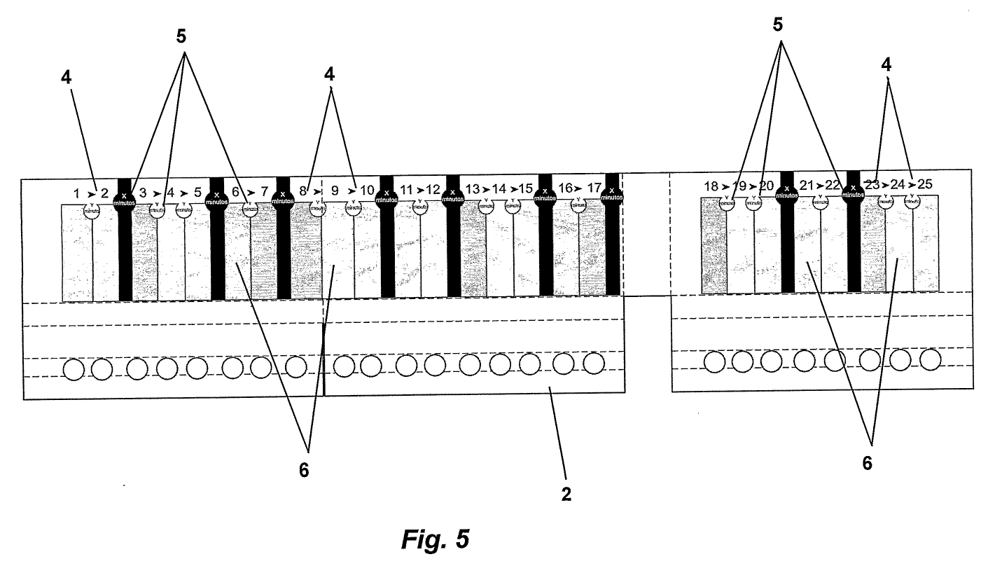

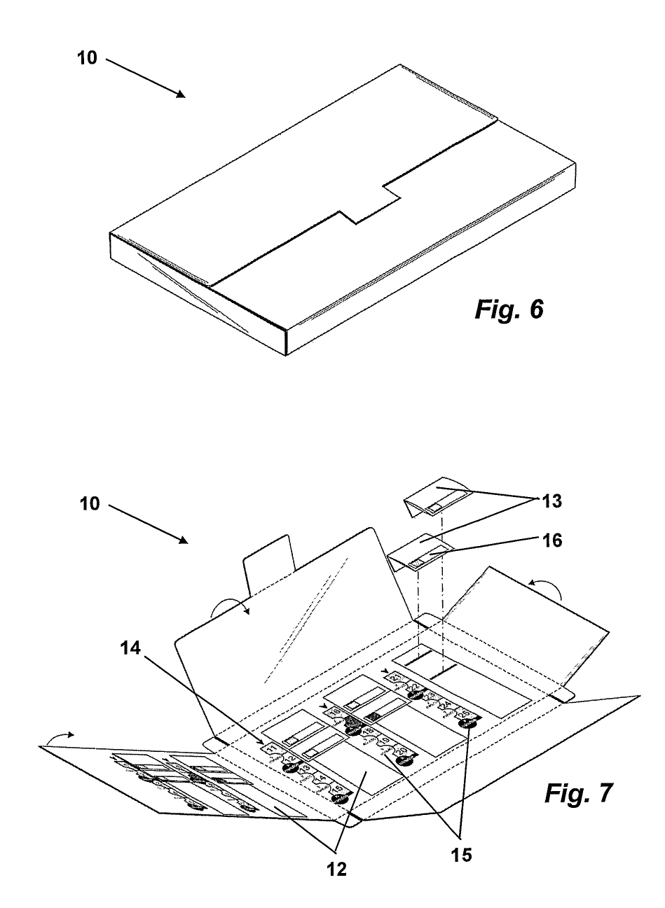

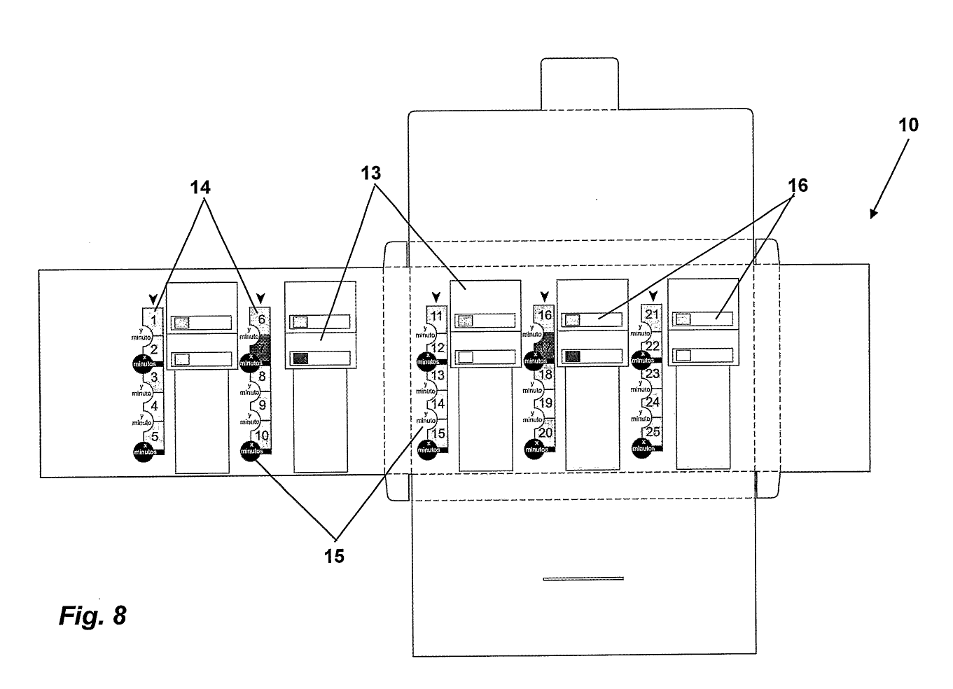

Therefore, kits (1, 10) present a specific sequence of groups comprising the composition: group 1, group 2, group 3, group 2, group 1, group 2, group 3, group 2, group 1, group 2, successively, summing up 25 recipients, according to FIGS. 1 and 2.

Each storage element (3, 13) contains composition components, preferably in the form of five globules soaked with the medicinal composition. Said storage elements may be available in various colors, preferably each color specifically related to one element, summing up seven colors being repeated and alternated, completing 25 elements.

The first embodiment of the medicinal kit (1) of the present invention advantageously comprises the following characteristics:

a) one single vertically positioned body and a lower base (2) to hermetically fix closed storage elements (3) containing the medicines;
b) indications on the single body of the order of administration of the medicine (4), time interval between each administration (5) and the identification of contents of each element (6).

In the second embodiment of the medicinal kit (10) of the present invention, the 25 hermetically closed storage elements (13) are substituted by simpler and more economic elements, which are fixed to the internal part of the kit. However, both embodiments have identical content.

Said second embodiment of the kit advantageously presents the following characteristics:

a) one single body, inside which storage elements (13) containing medicines are fixed;
b) indications on the order of administration of the medicine (14), time interval between each administration (15) and the identification of contents of each storage element (16).

The present invention also relates to the method for administration of the homeopathic medicine. The composition is given by the kit as follows:

### a) Group 1:

Antimonium crudun/1 minute interval/Kali carbonicum

About 10 to 30-minute interval, according to the used power.

### b) Group 2:

Mercurius solibilis/1 minute interval/Sulphur/1 minute interval/Natrum muriaticum

About 10 to 30-minute interval, according to the used power.

### c) Group 3:

Aurum metallicum/1 minute interval/Ammonium muriaticum

About 10 to 30-minute interval, according to the used power.

### d) Group 2:

Mercurius solibilis/1 minute interval/Sulphur/1 minute interval/Natrum muriaticum

About 10 to 30-minute interval, according to the used power.

### e) Group 1:

Antimonium crudun/1 minute interval/Kali carbonicum

About 10 to 30-minute interval, according to the used power.

f) Group 2:
Mercurius solibilis/1 minute interval/Sulphur/1 minute interval/Natrum muriaticum

About 10 to 30-minute interval, according to the used power.

### g) Group 3:

Aurum metallicum/1 minute interval/Ammonium muriaticum

About 10 to 30-minute interval, according to the used power.

### h) Group 2:

Mercurius solibilis/1 minute interval/Sulphur/1 minute interval/Natrum muriaticum

About 10 to 30-minute interval, according to the used power.

### i) Group 1:

Antimonium crudun/1 minute interval/Kali carbonicum

About 10 to 30-minute interval, according to the used power.

### j) Group 2:

Mercurius solibilis/1 minute interval/Sulphur/1 minute interval/Natrum muriaticum

About 10 to 30-minute interval, according to the used power.

Preferably, for medicinal powers up to 5 MFC, administration intervals between the groups as defined above are about 10 minutes and, for medicinal powers from 6 MFC to 60 MMFC, administration intervals between medicinal groups are about thirty minutes. According to the present invention, higher medicinal powers require longer time interval between doses.

It is needed to underline that the effective treatment of a few diseases may be done with the administration of one or more kits containing reinforced or not reinforced composition, until a satisfactory result is obtained. Time interval between each kit is about 120 days to 160 days, preferably 100 days, depending on the clinical evaluation by the physician.

So to illustrate the time interval between each kit, but not limiting the present invention, a few treatments effected with the present composition can be mentioned. Chagas' disease, for instance, has been healed in rats with the administration of one single kit. Chronic migraines or emotional dysfunctions needed just one kit each 60 or 90 days, summing up three administrations. Acute profiles used just one kit. Chronic and very old profiles required one kit each 60 or 90 days to conclude the curing process.

In a way not limitative of the invention, in case the kit needs to be repeated by one patient, it should have higher power than the previous one for the following prescription.

The homeopathic method using self-organization factors object of the invention, since it no longer requires individualization, allows the employed formulation to be standardized, opening up the possibility of double blind analysis, effective to reproduce results, which is a basic criterium in science. Furthermore, the present invention brings in economy to the patient in various aspects, since homeopathic medicines are easily accessed at low cost.

The following examples may be mentioned to illustrate but not to limit the invention.

### EXAMPLE 1

For specific intoxication by organophosphorous, with acutely intoxicated rats at DL50 for Chlorpyrifos and Methamidophos and chronically intoxicated rats, born from previously intoxicated female rats with methamidophos, results prove the cure of 100% intoxicated rats for both poisons in a two-hour deadline, in groups of twenty rats, with the model presented in the kit composed by 25 components, at the medicinal power of 500 CH, as applied in a single dose. The result found by this experiment drastically contrasts with the one as observed in the control group, in which within twenty minutes 80% of said animals died.

On the other hand, higher efficacy results were noted for the control of Erlich Tumor, in animals treated with the kit of the present invention, as applied under repeated doses each four days at the medicinal power of 10 CH.

We can observe the recovery of all animals exposed to poisons

(different agrochemicals), showing that the treatment does not aim to treat specific intoxication, but acts directly towards restoring the internal equilibrium of the individual.

### EXAMPLE 2

A descriptive epidemiological study was effected in 11 patients to observe the efficacy and effectiveness of the homeopathic treatment in intoxication profiles by agrochemicals in patients away from exposure, previously treated alopathically and which did not present favorable clinical response over immunological and neurological dysfunctions. The kit of the present invention was given to each patient. They were employed under dynamization powers varying from 1 OCH to 2 MFC.

From the 11 patients, just one patient has not returned for the second evaluation. Other evaluated patients improved about 70% of symptoms within the first four months of evaluation. Considerable improvement in the general state and emotional symptoms of these patients has been observed with good clinical response for digestive and respiratory symptoms. Positive response has also been observed for symptoms concerning immunological alterations, such as strong allergic profiles, as well as phobia profiles, mental confusion, depression, palpitation, tachycardia, dizziness, tip paraesthesia, arthralgia, myalgia and panic syndrome. These symptoms correspond to the main symptoms concerning this class of intoxication. In this period, however, slight improvement was obtained for specific symptoms such as cloudy vision, lack of memory and difficult concentration.

### EXAMPLE 3

A descriptive epidemiological study was developed in 11 patients, working for large chemical industries, victims of intoxication by solvents, especially toluene, benzene and xylene. Said exposed individuals present serious systemic compromising profiles, usually irreversible, with emphasis in immunological alterations and neurotoxic effects caused by said chemicals. Symptoms are characterized by psychic, behavior and motor alterations, manifesting days or months after exposure.

Said study had the general purpose to investigate chronic exposure to solvents and evaluate clinical indication for the use of homeopathics in irreversible chronic cases.

Patients as treated herein were also previously treated alopathically and have not presented favorable clinical response concerning immunological and neurological dysfunctions. The kit of the present invention was given to patients in dynamization powers varying from 6 D to 2 MFC.

All patients have returned to the second evaluation showing about 70% improvement in symptoms within the first four months of evaluation. Significant improvement was observed in symptoms such as dizziness, tip paraesthesia, palpitation, digestive symptoms, allergic dermatitis, dyspnea, cephalea, weight on legs with difficult deambulation, asthenia and prostration. In the emotional and mental area, considerable improvement in depression and panic syndrome symptoms, in the difficulty to process information, mental confusion, torpidity, strong behavior disorder with extremely aggressive profiles (even while sleeping), killing desires, hypersensitivity to any external stimulation, anxiety for fact anticipation and sensoperceptive alterations (expanding body sensation). We have however noticed that, within these first four months, patients disclosed slight improvement in symptoms such as persecutory delirium, strong loss of memory and reduction in concentration, with no response until now concerning symptoms such as buzzing and loss of hearing ability caused by such chemicals.

### EXAMPLE 4

We noticed in clinical research made with 89 patients, submitted to treatment with the kit under decreasing powers of 54 mM/50M/25M/200 CH that, despite presenting various different pathologies, they reported in 99% of the cases an improvement of their health profile and life quality.

### EXAMPLE 5

A 56-year old patient with history of hepatitis C for four years, having made therapy with interferon for two years with viral load remission. After six months, the profile suffered remission and therapy with Interferon had again been tried, with an expressive reaction of side effects to the treatment. After three months, new remission occurred, with increase in viral load to 280,000. A treatment with the kit containing the reinforced composition under the medicinal power of 10 CH in one single dosage has then been started and, one week later, its viral load had reduced to 5,000. A second dose of the same kit was prescribed on the seventh day under the medicinal power of 1 OCH and, after seven days, the viral load became negative. The same kit was later prescribed for seven more weeks, one dose per week, and there have been no further profile remissions.

### EXAMPLE 6

A 90-year old patient looked for homeopathic treatment with serious profile of zoster herpes on his face, intense asthenia and lack of appetite, with fever, and this profile evolved to erysipelas. The physician following him at that occasion suggested internment in the following morning, if no improvements in profile occurred. The kit containing the composition reinforced under power 10 CH was prescribed and, in the following morning, herpes had improved in 80% and the patient awoke with good disposition, no fever and all normal functions. A second prescription was not required. Within 48 hours, the patient presented no symptoms.

### EXAMPLE 7

A five-year old child, with a profile of purulent otitis and high fever, lack of appetite and prostration. He was given the kit containing reinforced composition under the power of 30 CH, presented remission of fever within the first few hours, after which two episodes of diarrhea with later improvement in otitis. In approximately 36 hours, he had absolutely no symptoms.

### EXAMPLE 8

A patient looked for help for his cat, presenting terminal profile of lung lymphoma. On that occasion, the animal presented a few important neurological signals, sialorrhoea, protruded tongue for two years, refusing to eat within the last 48 hours. It had already been submitted to various chemotherapy sessions and was being followed by three veterinaries. Upon the request for help, the kit was prescribed under the power of 10 CH each seven days. After a few hours from the first prescription, the animal ate again, its vitality significantly improved and, at the end of the fourth dose, after one month, X-ray has shown full remission of the lung injury. Medication remained being applied weekly for more three months.

### EXAMPLE 9

A 63-year patient, with a serious profile of arthritis rheumatoid with intense pain on fingers and some fingers already presenting some deformation. The kit was prescribed under the power of 155 CH and the doses were progressively alleviated until full remission, which occurred in the third month after the sole prescription.

### EXAMPLE 10

A 65-year old patient, with history of prostate tumor and later excision of the organ two years ago. He had depression and alleged post-surgical sexual impotence. Surgeons and physicians convinced him it was impossible to have improvements due to the surgical act and he looked for help in homeopathics. The kit was prescribed under the power 10 CH each seven days and, twenty days after the prescription, his sexual functions came back to normal, despite he no longer had the prostate.

### EXAMPLE 11

A 23-year old patient looked for treatment with history of strong abdominal pain for fifteen days, similar to an acute abdomen profile, however not presenting at that occasion any change in X-ray, tomography and laboratorial tests. Within that period, she had four episodes of internment to control the pain and investigate the case. Symptoms had then progressively increased and she responded less and less to antiinflamatories and antispasmodics. Laparoscopy was then suggested and would be performed on the following day. At that time, support from homeopathic therapeutics was requested and the 14 mM/9M/114-CH kit was prescribed (medication with decreasing medicinal powers). After three hours, the patient presented significant relief from the profile and, after twelve hours, she had absolutely no symptoms. A second prescription was not required.

### EXAMPLE 12

An eight-year old child looked for homeopathic treatment presenting an important aphtha profile for thirty days, with no response to any medicine. He had asthenia and lack of appetite for 24 hours, reported intense pain throughout the esophageal route and presented important aphtha injuries throughout the oral mucosa and tongue.

One single administration of the kit containing the composition reinforced under the power 10 CH was prescribed and, after twelve hours (upon waking up in the following morning), no injuries were reported and he had absolutely no symptoms. A second prescription was not required.

### EXAMPLE 13

Patient with sleep dysfunctions, reporting sleeplessness and night terror due to intense nightmares. He was treated with the kit containing the reinforced composition under decreasing powers at 13 mM/9M/114-CH/10 CH, respectively. After seven days, he indicated that sleep had returned to normal with remission of undesirable symptoms.

### EXAMPLE 14

Patient with serious depression profile, prostration, death thoughts, asthenia, irritability and lack of patience with relatives, afraid to go out on the street and getting away from responsibilities. Deep aprehension. He was medicated with the kit containing the reinforced composition under power 200 CH, single dosage. There was improvement in the profile after two days, with significant change in humor, reporting improvements in depression. After fifteen days, he had absolutely no symptoms.

### EXAMPLE 15

A 67-year old patient looked for homeopathic treatment since his case had been given up by thirteen physicians who gave him a life expectation of just three months due to a prostate tumor of more than 20 cm which had caused huge bone metastasis to various points of the rib, pelvis and femur. On that occasion, the patient had already lost about 15 kg and had intense pain profile due to bone injuries. There was no other alopathic medication to be given.

Daily prescription of the kit under the medicinal power of 6 CH was started and it was daily used for various months. After fifteen days, there was already full remission of pain and, after thirty days, the patient had already recovered his full normal weight.

PSA remission occurred, as it was initially at 280 and, after thirty days, was at 0.5 levels. Patient with six months of treatment presented no bone injuries and the prostate was absolutely normal.

### EXAMPLE 16

A 19-year old patient with history of bronchial asthma with serious episodes upon exposure to mould or post-virosis. He looked for treatment with a non-specific virosis profile, high fever and dry cough evolving to an important asthma profile. He was medicated with the kit containing the reinforced composition under power 30 CH and, after 24 hours, he had no fever and significant improvement in asthma. He had absolutely no symptoms after two days. He was subsequently medicated with the same kit each 15 days for two months and no longer presented any allergic or asthmatic symptom.

### EXAMPLE 17

A five-year old child presenting Kernicterus neurological profile due to post-parturition traumatism, started treatment with repetition pneumonia complaints for three years, with at least seven cases, besides repeated gastritis, otitis and amygdalitis, with intense agitation and very difficult psychomotor development.

He was medicated with the kit under the power 114 CH and presented full remission of all recurrent infection symptoms. After three months, he was medicated with the kit containing the reinforced composition under the medicinal power of 155 CH and, after six months, with the same kit at 157 CH. During that year, the patient did not present any further acute case and has presented considerable improvement in his neurological symptoms.

### EXAMPLE 18

A 22-year old female patient presents a serious case of acne as a consequence of micropolycystic ovariae, loss of hair, remitted sinusitis, fatigue, muscle pains, cephaleas and anemia.

She was medicated with the kit under the power 12 CH and presented no symptoms and perfect skin after sixty days.

### EXAMPLE 19

An 11-year old child traveled to a cold location and suffered virosis evolving into amygdalitis. The child remained for three days with no medication while traveling.

He was medicated with the kit under the power 5 MFC and presented immediate improvement in fever and general state, and in the following morning, i.e. eight hours later, no longer presented sore throat.

### EXAMPLE 20

A twelve-year old patient, under psychiatric treatment for one year, following medicinal therapy, had aggressive behavior, school inadequation, severe seborrhoea with hair loss and anorexia nervosa.

He was medicated with the kit under the power 42 MMFC and, already after the first prescription, seborrhoea fully disappeared and agressiveness was reduced, but he kept inadequate behavior and anorexia. Upon the second prescription, there was full improvement in the mental profile, and he returned to feed normally. The withdrawal of psychiatric medicine was forecasted to occur within a month.

The profile has been solved within eight months.

### EXAMPLE 21

A 42-year old patient, with history of traumatic injury on the right knee for about 18 years, which was not submitted to appropriate treatment. The profile evolved towards advanced arthrosis, loss of cartillage and a chronic disruption on the foremost crossed ligament was found. Surgery was made (articular hair-cutting+ligament fixation) in April 2005. Since then, his pain got considerably worse despite physiotherapeutics, causing numerous supressions with antiinflamatories.

He was medicated with the kit under the power 12 CH and progressive improvement in pain was noticed, with full cessation after fifteen days. Improvement in sleep, which was uneasy, was also verified, as well as better disposition.

### EXAMPLE 22

A two and a half-year old child, triplet and born from an early parturition, with considerable delay in motor development (he walked when two year and one month old) and walks with difficulty (like a drunk man). He presents repeated movements with arms shaking things, he does not interact with the world, does not reply to any request, does not play with other children nor with the other triplets, giving the impression that he does not understand what was spoken and what was asked from him. Completely absent.

He was medicated with the kit on power 3 MFC and, three months later, the child walks better and is much more alert. He understands and replies to commands, which never happened before. He stopped shaking things and hands and already says a few words (mum and Barney). At school, he already takes part of all activities whenever required. He had never interacted with the group before. He now interacts with everybody, making it clear that he perfectly understands what is requested from him and fights for his space with other children. He is still using diapers.

### EXAMPLE 23

15-month child, with high fever, prostration and difficult breathing. Auscultation revealed snoring, whistles and cracklings on the right side. Chest X ray showed bronchopneumonia on the right side.

He was medicated with the kit under power 50 CH and presented improvement of the whole profile within twelve hours, being auscultated again after 24 hours, showing lack of cracklings or whistles, rare snoring and no prostration.

### EXAMPLE 24

Male six-year old child presents severe asthma since one year old. Reports on intolerance to lactose, sporadic urticaria crisis, permanent rhinitis, difficulty to gain weight and create relationships. He arrived at the office with asthma crisis and significant bronchospasm with whistles on both lungs.

He was medicated with the kit under power 12 CH and already presented improvements during the use of the medicine, and the end of the crisis occurred within eight hours.

### EXAMPLE 25

Male 53-year old patient has severe migraines for four years, with daily and constant crisis which gets worse at night. He currently takes beta blockers, but giving little response.

He was medicated with the kit under power 33 MMFC and, six days after the start of the treatment, he presented just a few night occurrences of cephalea suspended with any analgesic. Previously, pains were not susceptible to common analgesics.

### EXAMPLE 26

Child presenting profile with 39° C. fever, adynamia and diffuse hyperemia in the throat.

He was medicated with the kit under power 200 CH-30 CH-10 CH in the morning, kept fever up to midnight with gradual decline of temperature during dawn. In the following morning, about twelve hours after medication, he presented no symptoms with improvement and no aggravation.

Considering the child's background, the profile would probably evolve to purulent amygdalitis. Homeopathics possibly aborted evolution.

### EXAMPLE 27

Four-year old child was sent to treatment due to strong stammer profile.

He was medicated with the kit under power 33 MMFC and, fifteen days later, his mother reported that stammer completely disappeared.

### EXAMPLE 28

Eleven-year old child with a gastroenteritis profile caused by rotavirus, presented diarrhea, vomiting, high fever and prostration.

He was firstly medicated with the kit under the power 14 MMFC, fever went down after eight hours, but on the following day he still presented four occurrences of diarrhea. He was again medicated with a kit with power 33 mM and, after this second series, diarrhea stopped within four hours.

### EXAMPLE 29

Female 52-year old patient presenting hypothyroidism profile with TSH 29.4, gain of weight, apathy, asthenia and prostration.

She was medicated with the kit under power 50 CH and after forty days, when tests were repeated, she verified that the TSH level was at 2.3, with improvement in all symptoms and normalization of the lipidogram which had high rates of LDL cholesterol and triglycerides.

### EXAMPLE 30

Female 25-year old patient, with history of a surgery to remove HPV injury since then presenting weekly relapses, occurring after acid application. There were numerous relapses.

She was medicated with the kit under the power 10 CH and presented no further relapses after seven days. Within three months, HPV was negative and keeping negative. The patient also presented improvement in allergic rhinitis, increase in concentration and bowel regulation.

### EXAMPLE 31

Female 40-year old patient presenting repetition urinary infection, therapy with antibiotics etc. with no results for two years. During consultation, the pacient presented important pain during urination with blooding. She brought the EAS test with result confirming the infection with more than 1,000,000 colonies of E. coli.

She was medicated with the kit under power 9 D and, six hours later, the series no longer presented blooding, but she still suffered pains during urination. Twelve hours later, she had no pains and no blooding.

### EXAMPLE 32

Female 82-year old patient looked for treatment with a chronic labyrinthitis profile which no longer responded to alopathic medication.

She was medicated with the kit under power 12 CH and, within ten days, was aleady with absolutely no symptoms and significant improvement of her general state.

### EXAMPLE 33

Female 32-year old patient with depression crisis, much fear paralysing her life and crisis which got worse at night, with the sensation of imminent death. She follows treatment with antidepressives and improves during their use.

She was medicated with the kit under the power 42 MMFC. After the series, she also had crisis of panic within longer intervals after two months from medication. Six months later, the patient had considerable improvement in her mental state, she is more confident and practically has no crisis.

### EXAMPLE 34

Patient presenting intense cold profile, strong adynamia, pharyngitis, myalgias and dry cough.

She was medicated with the kit under the power 500 CH but evolved with low improvement and the appearance of paroxysmal cough, low fever and pain in maxillary regions. Considering the evolution, a new kit with power 5M was given. Feeling of improvement was noticed soon after the end of the series. On the following day, i.e. after 12 to 24 hours, the patient presented just mild cough and light dysphonia.

### EXAMPLE 35

Six-month child presenting chronic catarrhal profile, fever and virus profile.

She was medicated with the kit under the power 5 MFC. The evolution to full improvement of the profile happened about 18 hours after treatment administration. Twenty days later, the child remains with no symptoms.

### EXAMPLE 36

Ten-year old boy, with pneumonia profile, sharp start of high fever with difficult breathing and pain while breathing, can only lie down on the opposite side to the pain. During auscultation, sterterous breathing on E base. He presented vomiting and refused to eat.

He was medicated with the kit under the power 10 MMFC. Pain and general state improvement occurred on the following day to medication, he returned to eat and no longer vomited. The boy had no fever after 48 hours.

### EXAMPLE 37

68-year old lady with CVA sequels, important language and memory alterations and macular injuries on MIE skin and scheduled biopsis.

She was medicated with the kit under the power 10 CH and spoke more freely within one month, forming phrases and had more active memory. Biopsis was taken out of schedule, since skin injuries had disappeared.

### EXAMPLE 38

One year and nine month old child with fever and vomiting profile for 48 hours, followed by diarrhea.

He was medicated with the kit under the power 4 MFC. Vomiting stopped after the series and the diarrhea within twelve hours.

### EXAMPLE 39

56-year old female patient presented crisis on the sciatic nerve, feeling intense pain for three days and difficult deambulation.

She was medicated by late afternoon with the kit under the power 33 MMFC and was 70% better in the morning. She had no symptoms after 24 hours.

### EXAMPLE 40

74-year old female patient with history of glaucoma for at least fifteen years.

She was medicated with the kit under the power 12 CH and after three months, when the evaluation was made again, she had no further alteration in eye pressure.

### EXAMPLE 41

Female 54-year old patient with history of blood hypertension oscilating between maximum 18 and 16 when no anti-hypertensives were used.

She was medicated with the kit under the power 50 CH and within three months the patient returned stating she could no longer take anti-hypertensives, since she presented hypotension, keeping a pressure of 9/6 mmHg.

### EXAMPLE 42

Male four-year old child with important rhinitis, much lachrymation and intense lack of air, followed by oedema on tonsils. The child is very excited, but in school he presents strange behavior and cannot interact with other children, nor correctly interact with teachers.

He was medicated with the kit under the power of 1 MFC. The child evolved well since the first dosage, but after the last one he no longer presented rhinitis crisis, tonsil became much smaller and he started to be sociable at school.

### EXAMPLE 43

Patient presenting irritable colon with colics, much flatulence, lumbalgia and migraine. She also started diarrhea with much colic, violent vomiting but no fever.

She was medicated with the kit under the power FAO 1 MFC and presented improvement within about 24 hours.

### EXAMPLE 44

Seventy-year old male patient, after efforts, presented intense pain throughout his arm, irradiating from the shoulder to the pulse which could not be reduced by using antiinflamatories. After twelve days, FAO was prescribed.

He was medicated with the kit under the power 1 OCH. The patient reported that the pain was immediately released and he immediately recovered movements. Within two days, he had absolutely no symptoms.

### EXAMPLE 45

Teenager presenting dermatitis profile evolving to hypochromic injuries similar to vitiligo. The profile persisted for two years and did not respond to corticotherapy. Some dermatologists diagnosed it as a form of psoriasis.

He was medicated with the kit under the power 7 CH. Within fifteen days, injuries had already improved in 80%, and the injuries fully disappeared within thirty days.

### EXAMPLE 46

Three-year old female child had been treating atopic eczema since she was a baby with classic homeopathics, but always presented dry skin, much coryza and nose secretion, with some occurrences of severe urticaria and much itching throughout the body.

She was medicated with the kit under the power 33 MMFC and, after four months, the child had fully clean skin with no itching, no drying, no constant VAS secretions, sleeping much better and emotionally very well.

### EXAMPLE 47

55-year old patient feeling strong pain for sixty days due to spur, with the pain irradiating to the groin of the same leg. The patient also had prostration and submissiveness for life.

She was medicated with the kit under the power 33 MMFC. The patient reported that, from the third day, she started to become much better and now has no symptoms.

### EXAMPLE 48

24-year old female patient presented, after a profile of amygdalitis treated with amoxilin, return of high fever, followed by articular pain on wrists quickly evolving to the shoulders. Urgent laboratorial tests were compatible with rheumatic fever.

She was medicated with the kit under the power 10 MFC. The fever went down two hours after the series and, within six hours, there was no further pain in any joint. The possibility of endocarditis was researched but discharged. Laboratorial tests within 24 hours already presented considerable improvement of the profile.

### EXAMPLE 49

42-year old patient with history of myomas and endometriosis for five years. The patient had been much handled and evolved with retention of menstrual flow for spasm on the uterine colon, causing much colic which can only be solved with drainage.

She was medicated with the kit under the power 6 CH and presented spontaneous blooding 22 hours after medication. The patient reports being much better and happy.

### EXAMPLE 50

72-year old patient in the hospital with a serious dehydration profile by diabetes (hyperosmolar syndrome). A profile of intense muscle weakness was noticed, compromising muscles of lower and higher limbs, face (palpebral ptosis), tongue (defective speech) and oropharinx (difficult swallowing and secretion elimination). She needed aerial route aspirations and feeding by enteral probe. Chest radiography suggests LID tumoral injury.

She was medicated with the kit under the power 12 CH. Improvement in swallowing and secretion elimination ability was noticed within 48-72 hours, allowing the probe to be taken out and the administration of oral diet; secretions were not retained on the pharynx (effective cough). The patient was also in good humor. The patient had prevision to leave the hospital with clinical review at pneumology and medical clinics—homeopathics (probable tumor with paraneoplasic myasthenic syndrome).

1. MEDICINAL COMPOSITIONS, which comprises the following homeopathic elements: Antimonium crudun, Kali carbonicum, Mercurius solubilis, Sulphur, Natrum muriaticum, Aurum metallicum and Ammonium muriaticum, under dynamization ranges from 6 D to 50 D, 6 CH to 999 CH, from 6 LM to 200 LM or from 1 MFC to 60 MMFC (continued flow) in a 30% hydroalcohol solution to obtain 100% diluted solution.

2. MEDICINAL COMPOSITIONS of claim 1, wherein the homeopathic elements are repeated in medicinal compositions.

3. MEDICINAL COMPOSITIONS of claim 1, wherein the components are repeated in a sequence order as follows:
1) 0.20 ml of the substance Antimonium crudun in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
2) 0.20 ml of the substance Kali Carbonicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
3) 0.20 ml of the substance Mercurius solubilis in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
4) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
5) 0.20 ml of the substance Natrum muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
6) 0.20 ml of the substance Aurum metallicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
7) 0.20 ml of the substance Ammonium muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
8) 0.20 ml of the substance Mercurius solubilis in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
9) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
10) 0.20 ml of the substance Natrum muriaticum in dynamizations within the following ranges: from 6 D to 50 D, from 6 CH to 999 CH, from 6 LM to 200 LM or from 1 MFC to 60 mMFO (continued flow) under 30% hydroalcohol solution to obtain 100% diluted solution.
11) 0.20 ml of the substance Antimonium crudun in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
12) 0.20 ml of the substance Kali Carbonicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
13) 0.20 ml of the substance Mercurius solubilis in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
14) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
15) 0.20 ml of the substance Natrum muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
16) 0.20 ml of the substance Aurum metallicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
17) 0.20 ml of the substance Ammonium muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
18) 0.20 ml of the substance Mercurius solubilis in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
19) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
20) 0.20 ml of the substance Natrum muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
21) 0.20 ml of the substance Antimonium crudun in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
22) 0.20 ml of the substance Kali Carbonicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
23) 0.20 ml of the substance Mercurius solubilis in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
24) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
25) 0.20 ml of the substance Natrum muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.

4. MEDICINAL COMPOSITIONS of claim 1, wherein the medicinal potentialization varies from 6 D to 100 CH for the treatment of serious profiles such as Zoster herpes, hepatitis C with very high viral load, collagenosis, rheumatic profiles, Parkinson, hypothyroidism and hyperthyroidism, prostatic hyperplasia, ulcerative rectum colitis, tendinitis gravis, repeated effort injuries, migraines, neuropathies, encephalitis, fibromyalgias, atopic dermatitis, asthma, bronchitis, serious eczema, gastric ulcera, duodenitis, hepatitis B, hepatitis A, gastroesophagus reflow, strong painful profiles, genetic origin diseases such as Huntington's disease and kidney insufficiency.

5. MEDICINAL COMPOSITIONS of claim 4, wherein the medicinal potentialization is 9 D.

6. MEDICINAL COMPOSITIONS of claim 4, wherein the medicinal potentialization for the treatment of chronic and serious profiles such as colagenosis and degenerative diseases is 6 D or 1 OCH.

7. MEDICINAL COMPOSITIONS of claim 4, wherein the medicinal potentialization for the treatment of migraines, cephalea and pain profiles in general is 6 D, 9 D, 30 D, 10 CH.

8. MEDICINAL COMPOSITIONS of claim 1, wherein the medicinal potentialization varies from 9 D to 15 MFC for the treatment of severe constipation profiles, acute and chronic diarrheas, urinary infections, rhinitis, sinusitis, bad digestion, non-specific virosis, bacterial infections, cardiovascular diseases, blood hypertension, tropical diseases, non-specific allergic profiles, amygdalitis, otitis, sinusitis, conjunctivitis and bronchial asthma.

9. MEDICINAL COMPOSITIONS of claim 8, wherein the medicinal potentialization varies from 155 CH to 15 MFC.

10. MEDICINAL COMPOSITIONS of claim 9, wherein the medicinal potentialization is 5 MFC.

11. MEDICINAL COMPOSITIONS of claim 8, wherein the medicinal potentialization for the treatment of acute and infectious profiles is 6 D or 50 D, 5 MFC or 10 MFC; and 6 CH or 500 CH.

12. MEDICINAL COMPOSITIONS of claim 8, wherein the medicinal potentialization for the treatment of acute and chronic allergic profiles, such as bronchic asthma, rhinitis and sinusitis varies from 6 D to 50 D; 6 CH or 500 CH; and from 1 MFC to 15 MFC.

13. MEDICINAL COMPOSITIONS of claim 12, wherein the medicinal potentialization is 12 D.

14. MEDICINAL COMPOSITIONS of claim 1, wherein the medicinal potentialization for the treatment of profiles with emotional dysfunctions varies from 9 D to 15 MFC.

15. MEDICINAL COMPOSITIONS of claim 14, wherein the medicinal potentialization varies from 9 D to 50 D.

16. MEDICINAL COMPOSITIONS of claim 15, wherein the medicinal potentialization is 12 D.

17. MEDICINAL COMPOSITIONS of claim 1, wherein the medicinal potentialization for the treatment of agrochemical intoxication varies from 9 D to 155 CH.

18. MEDICINAL COMPOSITIONS of claim 1, wherein the medicinal potentialization varies from 9 D to 60 MMFC for the treatment of reactive depression, dysthymia, loss of libido, prostration, apathy, memory dysfunctions, anxiety, obsessive compulsive dysfunctions and phobia profiles such as panic syndrome.

19. MEDICINAL COMPOSITIONS of claim 18, wherein the medicinal potentialization varies from 9 D to 500 CH.

20. MEDICINAL COMPOSITIONS of claim 19, wherein the medicinal potentialization is 12 D.

21. MEDICINAL COMPOSITIONS of claim 1, wherein the medicinal potentialization for the treatment of non-specific virus profiles varies from 9 D to 10M.

22. MEDICINAL COMPOSITIONS of claim 1, wherein the medicinal potentialization for the treatment of gynecological diseases varies from 9 D to 5 MFC.

23. MEDICINAL COMPOSITIONS of claim 1, wherein the medicinal potentialization for the treatment of blood hypertension varies from 9 D to 200 CH.

24. MEDICINAL COMPOSITIONS of claim 1, wherein the medicinal potentialization for the treatment of endocrine dysfunctions varies from 6 CH to 5 MFC.

25. MEDICINAL COMPOSITIONS of claim 1, wherein the medicinal potentialization for the treatment of circulatory dysfunctions varies from 6 D and 150 CH.

26. MEDICINAL COMPOSITIONS of claim 1, wherein the medicinal potentialization for the treatment of respiratory dysfunctions varies from 9 D to 200 CH.

27. USE OF MEDICINAL COMPOSITIONS, as defined in any of claims 1 to 26, being for the production of a homeopathic medicine.

28. USE of claim 27, being for the production of a homeopathic medicine which varies in power according to specific clinic cases.

29. MEDICINAL KIT containing medicinal compositions as defined in claim 1, which comprises 25 storage elements (3, 13), each one containing respectively:
1) 0.20 ml of the substance Antimonium crudun in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
2) 0.20 ml of the substance Kali Carbonicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
3) 0.20 ml of the substance Mercurius solubilis in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
4) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
5) 0.20 ml of the substance Natrum muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
6) 0.20 ml of the substance Aurum metallicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
7) 0.20 ml of the substance Ammonium muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
8) 0.20 ml of the substance Mercurius solubilis in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
9) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
10) 0.20 ml of the substance Natrum muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
11) 0.20 ml of the substance Antimonium crudun in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
12) 0.20 ml of the substance Kali Carbonicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
13) 0.20 ml of the substance Mercurius solubilis in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
14) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
15) 0.20 ml of the substance Natrum muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
16) 0.20 ml of the substance Aurum metallicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
17) 0.20 ml of the substance Ammonium muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
18) 0.20 ml of the substance Mercurius solubilis in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
19) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
20) 0.20 ml of the substance Natrum muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
21) 0.20 ml of the substance Antimonium crudun in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
22) 0.20 ml of the substance Kali Carbonicum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
23) 0.20 ml of the substance Mercurius solubilis in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
24) 0.20 ml of the substance Sulphur in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.
25) 0.20 ml of the substance Natrum muriaticum in dynamizations within the following ranges: from 6 D to 50 D, from 6 CH to 999 CH, from 6 LM to 200 LM or from 1 MFC to 60 MMFC (continued flow) under 30% hydroalcohol solution to obtain 100% diluted solution.

30. MEDICINAL KIT for storage of medicinal compositions as defined in claim 1, which comprises:
a) one single vertically positioned body and a lower base (2) to fix hermetically closed storage elements (3) containing the medicines;
b) indications on the single body of the order of administration of the medicine (4), time interval between each administration (5) and the identification of contents of each element (6).

31. MEDICINAL KIT for storage of medicinal compositions as defined in claim 1, which comprises:
a) one single body, inside which storage elements (13) containing medicines are fixed;
b) indications on the order of administration of the medicine (14), time interval between each administration (15) and the identification of contents of each storage element (16).

32. KIT of any of claims 30 or 31, wherein the storage elements (3, 13) summing up 25 elements, each one containing five globules soaked with the medicinal composition.

33. KIT of claim 32, wherein said storage elements (3, 13) have different colors, each color specifically related to one element summing up seven elements repeating and alternating to perform the 25 storage elements.

34. KIT of any of claims 30 or 31, which comprises a specific sequence of the groups comprised by the composition: group 1, group 2, group 3, group 2, group 1, group 2, group 3, group 2, group 1, group 2, successively.

35. METHOD FOR ADMINISTRATION OF MEDICINAL COMPOSITIONS, as defined in claim 1, wherein each medicinal composition is inserted in each recipient of the same Group being administered within a time interval of one minute and between different Groups within a ten-minute interval, also being said administration followed by the consecutive indication as established in the kit as per claims 29 to 34.

36. METHOD of claim 35, wherein said medicinal compositions are administered by sublingual route.
